(19) United States
(12) Patent Application Publication
(10) Pub. No.: US 2026/0063541 A1
(43) Pub. Date: Mar. 5, 2026

(54) SYSTEMS AND METHODS FOR MEASURING PROPERTIES OF WATER AT SITE

(71) Applicant: HYDRO-METRICS LIMITED, Christchurch (NZ)

(72) Inventor: David Michael Rankin, Lincoln (NZ)

(21) Appl. No.: 19/108,300

(22) PCT Filed: Sep. 5, 2023
(86) PCT No.: PCT/IB2023/058749

§ 371 Date: Mar. 3, 2025

(30) Foreign Application Priority Data
Sep. 5, 2022 (AU) 2022902557

(51) Int. Cl. G01N 21/33; G01N 21/31; G01N 33/18

(52) U.S. Cl. CPC G01N 21/33; G01N 21/314; G01N 33/18; G01N 2021/3155

(57) ABSTRACT

There is described a system and method for measuring properties of water, such as surface water at site. That site may be in an agricultural setting. The properties of water, such as surface water may include nitrates and/or dissolved organic carbons. An example system may comprise a light source configured to emit a broadband source signal comprising a particular bandwidth for use in measuring a range of particular properties of water as well as an optical device configure to split the source signal into a measurement signal, and a corresponding reference signal. The system may also comprise a sensor unit configured to communicate a measurement signal through water at the sensor unit (e.g., when located in a stream), and a spectrometer configured to receive both a measurement signal having been communicated through water at the sensor unit and a reference signal from the optical device. Such a spectrometer may be configured to separate received measurement signals and reference signals into common component wavelengths, those common component wavelengths being associated with expected properties of water; and the system may be further configured to use respective component wavelengths of a measurement signal together with those of a reference signal to determine one or more particular properties of measured water.

### CROSS-REFERENCE TO RELATED APPLICATIONS

The present application is a U.S. National Stage application, filed under 35 U.S. C. § 371, of International Application No. PCT/IB2023/058749, filed on Sept. 5, 2023, which claims the benefit of Australian provisional patent application Ser. No. 2022902557, filed on Sept. 5, 2022, each of which is hereby incorporated herein by reference in its entirety.

### TECHNICAL FIELD

There are described systems, methods and associated data for measuring properties of water at site, such as at an agricultural site. In some examples, the systems, methods and data may be usable when measuring surface water and/or ground water, which may have multiple unknown components (e.g., contaminants).

### BACKGROUND

Water monitoring continues to be of increasing importance when considering land and water management. Ensuring pollution is minimized and/or excessive over application of chemicals in agricultural settings is avoided is important to many land managers and businesses. This is particularly true when considering waterways or the like that may be affected in rural agricultural settings by runoff of fertilizers and/or pesticides. In such settings, excessive nitrates and other chemicals may find their way into the water which can cause significant environmental and human health issues, as well as being indicative of other management issues such as general overuse of chemicals, poor soil health or drainage at site, which may need attention. Industrial and/or human activity may also drive runoff problems. There continues to be a need for accurate measurement and monitoring of contaminants in groundwater and/or surface water in such environments, and in particular such measurement and monitoring occurring in real time.

Such settings are however often remote and in rugged locations, and can experience adverse or extreme weather conditions. Further, ease of access can prove challenging when considering any such monitoring, or maintenance of equipment at site. Further still, the particular type of contaminant or composition of the water at site may be unknown.

There continues to be a need for accurate measurement and monitoring of surface water and/or groundwater in such environments, particular real-time monitoring and/or longer term monitoring (e.g., trend measurement and/or monitoring). Such monitoring may benefit from being cost effective, robust and/or any equipment being easily usable and/or calibrated. It may additionally or alternatively be valuable to provide systems or methods for monitoring one or both of surface water and groundwater.

### SUMMARY

There are described systems, methods and data, including databases, for measuring properties of water at site (e.g., surface water at site), such as at an agricultural site. The systems, method and data described may be usable for real time and/or longer-term monitoring (e.g., trend measurement and/or monitoring). The systems, methods and data described may be usable so as to be accurate, cost effective, robust and/or easily usable and/or calibrated. It may be that the systems, method, etc., are additionally or alternatively used to monitor one or both of surface water and groundwater. It may be that the systems, method, etc., additionally or alternatively at least provide the public with a useful alternative.

In one example, there is described a system for measuring properties of water (e.g., surface water) at site. The system may be specifically configured to measure nitrates and/or dissolved organic carbons in water. The system may be configured to measure total suspended solids (TSSeq), Chlorine, Bromine, or metals.

Such a system may comprise a light source (e.g., flash bulb) configured to emit a broadband source signal comprising a particular bandwidth for use in measuring a range of particular properties of water. The light source may be configured to emit a broadband source signal comprising wavelengths between 190 nm and 420 nm.

The system may comprise an optical device, for example an optical coupler (e.g., in one example having a coupling ratio of approximately 1:1 (50:50)). The optical device may be configured to split a source signal into a measurement signal, and a corresponding reference signal. The system may comprise a sensor unit configured to communicate a measurement signal through water at the sensor unit. The system may comprise a spectrometer configured to receive both a measurement signal having been communicated through water at the sensor unit and a reference signal from the optical device. Such a spectrometer may be configured to separate received measurement signals and reference signals into common component wavelengths. Those common component wavelengths may be associated with expected properties of water. The system may be configured to use (e.g., for calculation purposes) respective component wavelengths of a measurement signal together with those of a reference signal to determine one or more particular properties of measured water.

In some examples, the spectrometer may comprise a diffraction grating. The diffraction grating may be configured to separate first and/or second signals (e.g., a received measurement signal and a received reference signal) into their component wavelengths. The diffraction grating may be configured as a concave grating. The grating may be configure to be symmetrical and having a principal optical focal plane. Each of a received measurement and reference signals (e.g., first and second signals) may be projected toward the grating along an off-axis focal line (e.g., towards a center region and/or point of the grating). The off-axis focal line of a first signal (e.g., measurement signal) may be different from an off-axis focal line of a second signal (e.g., reference signal).

The spectrometer may be configured such that a measurement signal is projected along a first off-axis focal line, and a reference signal is projected along a second off-axis focal plane, and wherein the first and second off-axis focal line are positioned either side of the principal focal plane of the grating.

The spectrometer may comprise a first sensor array (e.g., measurement sensor array) and a second sensor array (e.g., reference sensor array). Each array may be configured to receive common component wavelengths of (first and/or second) measurement and reference signals having been diffracted from the grating. The system may be configured such that the sensor arrays are positioned off-axis from the principal plane of the diffraction grating.

Further, one or both of the sensor arrays may be adjustable relative to principal plane of the diffraction grating (e.g., adjustable within housing). The diffraction grating may additionally or alternatively adjustable so to provide the adjustment relative to the principal plane.

The spectrometer may comprise a filter arrangement, e.g., positioned over certain portions (e.g., some or all) of the measurement sensor array and the reference sensor array. The filter arrangement may be configured to filter some of the wavelength components from measurement and reference signals received at the arrays.

The system may be configured such that the spectrometer receives both a measurement signal and a reference signal simultaneously or essentially simultaneously for a particular emission from the light source.

The system may be configured such that the spectrometer (e.g., diffraction grating of the spectrometer) separates the received measurement signal into a plurality or multiple component wavelengths simultaneously. The system may be configured such that the spectrometer (e.g., diffraction grating of the spectrometer) separates the received reference signal into a plurality or multiple component wavelengths simultaneously. The system may be configured such that the spectrometer (e.g., diffraction grating of the spectrometer) separates the received measurement signal and reference signal into a plurality or multiple component wavelengths (e.g., the common component wavelengths) simultaneously.

The sensor unit may comprise a slotted gap, within which water is located in use, and through which a measurement signal is communicated. The sensor unit may be configured such that an opening of the slotted gap is directed downwardly when positioned in water, in use. The sensor unit may be configured to be buoyant in water (e.g., surface water) so as to be positioned, in use, at a generally fixed location below the surface of a body of water.

The system may comprise a housing containing the light source and the spectrometer, and wherein the sensor unit is removably connected to the housing via communication link.

The system may be configured to use measured component wavelengths of a measurement signal and a reference signal, together with ancillary data, in order to determine properties of measured water. Such ancillary data may comprise data associated with the location of the system, the time of measurement, temperature and/or properties of the measured water matrix. The system may comprise, or be in communication with, a database comprising a plurality of calibration datasets for use in determining properties of measured water based on measurement and reference signals. The system may be configured to select one or more particular calibration datasets for use, based on the ancillary data.

In some examples, there is described a method for measuring properties of water (e.g., surface water) at site. The method may measure nitrates and/or dissolved organic carbons in water. The method may measure total suspended solids (TSSeq), Chlorine, Bromine, or metals.

The method may comprise emitting a broadband source signal comprising a particular bandwidth for use in measuring a range of particular properties of water. The method for example may comprise emitting a broadband source signal comprising with a bandwidth of wavelengths between 190 nm and 420 nm.

The method may comprise splitting the source signal into a measurement signal, and a corresponding reference signal. The method may comprise communicating the measurement signal through water (e.g., surface water). The method may comprise receiving both the measurement signal, having been communicated through water, and the reference signal, and separating the measurement signal and reference signal into common component wavelengths. Such common component wavelengths may be associated with expected properties of water. The method may comprise using respective component wavelengths of the measurement signal together with those of the reference signal to determine one or more particular properties of measured water.

The method may comprise separating the received measurement signal into a plurality or multiple component wavelengths simultaneously. The method may comprise separating the received reference signal into a plurality or multiple component wavelengths simultaneously. The method may comprise separating the received measurement signal and reference signal into a plurality or multiple component wavelengths (e.g., the common component wavelengths) simultaneously.

In some examples, there is described a method for measuring properties of water at site where the method comprises using received component wavelengths of a measurement signal together with received common wavelength components of a reference signal, those common component wavelengths being associated with expected properties of water, the measurement signal having been passed through water, and the reference signal and measurement signal having been split from a particular emission of a broadband source signal comprising a particular bandwidth, for use in measuring a range of particular properties of water.

In some examples, there is a computer program product usable to provide the methods described (e.g., a computer readable medium carrying instructions which, when executed by at least one processor of a system, cause the system to carry out the methods described).

In some examples, there is described one or more databases, e.g., comprising one or more calibration datasets for use with the system or method described herein. Such datasets may be usable together with the respective component wavelengths of a measurement signal and reference signal in order to determine one or more particular properties of measured water. Some or all of the calibration datasets may include data associated with location and/or temperature, for that particular dataset.

In some examples, there is described a method comprising installing a system according to the embodiments described herein at a particular site. In those cases, the sensor unit may be installed in water and the spectrometer may be installed remotely from the sensor unit. The method may comprise operatively connecting the sensor unit to the spectrometer using a communication link (e.g., one or more fibers).

In some examples, there is described a method of replacing modular components of a system according to the embodiments described herein. The method may include at site, removing and/or replacing one or more of the light source, optical device, sensor unit and/or spectrometer.

In some examples, there is described a further system for measuring properties of water (e.g., surface water) at site. The system may comprise a light source configured to emit a broadband source signal comprising a particular bandwidth for use in measuring a range of particular properties of water. The system may comprise a sensor unit configured to receive via a communication link (e.g., a single fiber link), from the light source, a measurement signal and communicate that measurement signal through water at the sensor unit. The sensor unit may be configured to reflect that measurement signal back across water for receipt and further transmission using the same fiber link or other type of communication link. The system may comprise a spectrometer configured to receive a measurement signal from the sensor unit, and to separate that measurement signal into component wavelengths, those component wavelengths being associated with expected properties of water.

Such a system may be configured to use respective component wavelengths of a measurement signal to determine one or more particular properties of measured water.

The system may be configured such that the spectrometer (e.g., diffraction grating of the spectrometer) separates the received measurement signal into a plurality or multiple component wavelengths simultaneously.

In some examples, the system may further comprise an optical device configure to split a source signal into a measurement signal, and a corresponding reference signal. The spectrometer may be configured to receive both a measurement signal from the sensor unit and a reference signal from the optical device. The spectrometer may be configured to separate measurement signals and reference signals into common component wavelengths. The system may be configured to use respective component wavelengths of a measurement signal together with those of a reference signal to determine one or more particular properties of measured water. The system may be configured such that the spectrometer (e.g., diffraction grating of the spectrometer) separates the received reference signal into a plurality or multiple component wavelengths simultaneously. The system may be configured such that the spectrometer (e.g., diffraction grating of the spectrometer) separates the received measurement signal and reference signal into a plurality or multiple component wavelengths (e.g., the common component wavelengths) simultaneously.

The term ‘comprising’ as used in this specification and claims means ‘consisting at least in part of’. When interpreting statements in this specification and claims which include the term 'comprising', other features besides the features prefaced by this term in each statement can also be present. Related terms such as ‘comprise’ and ‘comprised’ are to be interpreted in a similar manner.

To those of ordinary skill in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

These and other objects, advantages, purposes and features of the present disclosure will become apparent upon review of the following specification in conjunction with the drawings.

### BRIEF DESCRIPTION OF THE FIGURES

FIG. 1 shows an example of a system for measuring properties of water, such as surface water at site;

FIGS. 2a-2c show an example of a sensor unit usable with the system of FIG. 1;

FIG. 3 shows an example of cleaning apparatus for use with the sensor unit of FIGS. 2a-2c;

FIG. 4 shows a further example of a sensor unit usable with the system of FIG. 1;

FIG. 5 shows an example of housing usable with the system of FIG. 1;

FIG. 6 shows an example of a light source usable with the system of FIG. 1;

FIG. 7 shows a simplified schematic of the system of FIG. 1, having a transmissive sensor unit;

FIG. 8 shows an alternative schematic of the system of FIG. 1, having a reflective sensor unit;

FIG. 9 shows an example of a spectrometer for use with the system of FIG. 1; and

FIGS. 10a and 10b show paths of a measurement signal within the spectrometer of FIG. 9.

### DETAILED DESCRIPTION

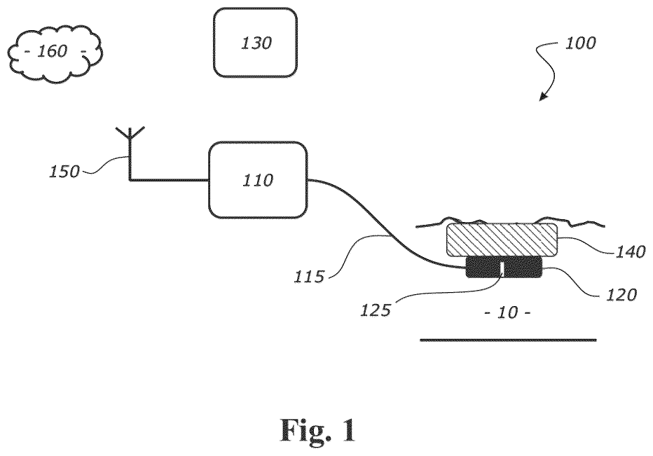

FIG. 1 shows an example of a system 100 for measuring properties of water 10, such as surface water at site. That site may be remote and/or associated with an agricultural setting where surface water 10 may be in the form of stream or other such waterway, and which may be affected by runoff from nearby land. Industrial and/or human activity may also drive runoff. It will be appreciated that the use of fertilizers, animal stocking and the use of other chemicals (e.g., pesticides) at or around the site may affect the properties or otherwise composition of the water 10, particularly surface water as opposed to groundwater. It will be appreciated that surface water in particular may contain a number of unknown contaminants (e.g., as opposed to ground water, which may be less affected by pollutants) as well as being somewhat variable due to the instream processes that occur, and the overall degree of connection to ground water.

In the following examples, the system 100 described is configured to measure nitrate (e.g., NO3-N, NO2-N) concentrations in water 10, e.g., surface water. It will readily be appreciated however that the system 100 may be configured to measure various additional or alternative compositions, including dissolved carbons, whether biologically active or not (e.g., CODeq (optically equivalent chemical oxygen demand), BODeq (optically equivalent biological oxygen demand), DOCeq (optically equivalent dissolved organic carbon), TOCeq (optically equivalent total organic carbon)), total suspended solids (TSSeq), Chlorine, Bromine, or metals (e.g., Silver), or the like. As will be described, in this particular example the system 100 is configured to measure spectral absorption characteristics of the water 10 in order to determine the properties of that water 10, and so to determine composition of nitrates, or the like. It will be appreciated in the following examples that the system 100 may be considered to be specifically configured to measure water 10 at site (e.g., surface water), which may have many unknown dissolved components and contaminants. In some examples, the system 100 may be configured to measure for the unknown concentration or presence of multiple different components, for example, at the same time (e.g., measuring for nitrate composition as well as dissolved organic carbon composition).

Shown in FIG. 1, the system 100 comprises a housing 110 and a sensor unit 120, connected to each other via a suitable communication link 115, which in this is example is a waveguide and more particularly an optical fiber. The link (or fiber) is configured to communicate signals between the sensor unit 120 and the housing 110 as will be described. For simplicity, this is shown in FIG. 1 as a single communication link 115, but of course it will be appreciated that this link 115 may comprise multiple communication paths (e.g., to and from the sensor unit 120), which may be along common or multiple fibers, as well as potentially comprising additional power and/or communication lines (some of which may be electrical or mechanical). As such, the link 115 in some cases may be a single or multiple fibers, and/or may include additional lines, which may be provided as part of a bundle or umbilical, or may be provided separately, as will be appreciated. In this example, the fiber link 115 is specifically configured to permit communication of optical signals comprises wavelengths of between 190 nm and 420 nm (e.g., comprising deep UV or visible wavelengths).

The sensor unit 120 is configured to be situated in a stream or other such waterway so that it is positioned below the surface of the water 10, whereas the housing 110 may be located away from the water 10 (e.g., on nearby land). In this example, the system 100 is configured such that sensor unit 120, in use, is positioned above the ground or bed of the water 10, while maintaining some or all of the sensor unit 120 in the water 10. In other similar words, the sensor unit 120 is configured to be maintained within the body of water 10 and this may be achieved by securing or fixing the location of the sensor unit 120 at site. However, as the level of water 10 rises and falls, such fixing may cause the sensor unit 120 to become exposed undesirably beyond the level of water 10. As such, in alternative examples, the system 100 may comprise a positioning aid 140 (e.g., a buoyancy device), configured to maintain the relative position of the sensor unit 120 and the level of water 10 (e.g., so as to be maintained at a known or approximated distance relative to the surface of the water 10).

Here, the system 100 further comprises a power source 130, which in this example is a photovoltaic (PV) unit. In other examples, the power source 130 may be configured to extract energy from the water 10, for example, or indeed from a local power network or the like. Multiple power sources may also be used. The system 100 in this example further comprises a communication arrangement 150 configured to communicate data to and/or from a remote site 160, as will be described. By way of an example that remote site 160 is illustrated in FIG. 1 as a cloud site. The system may be configured to communicate using cellular protocols, and/or other wireless network protocols (e.g., Wifi, wireless local area network (WLAN), etc.).

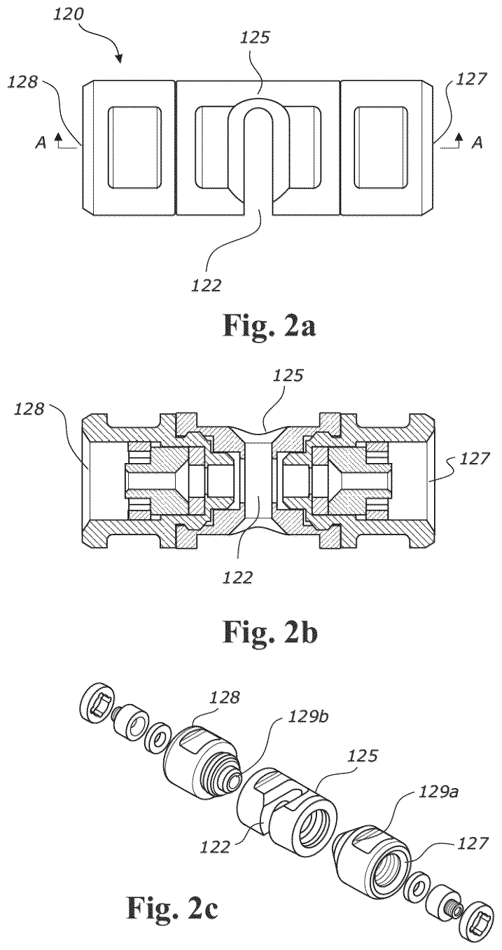

FIGS. 2a-2c shows one example of the sensor unit 120 in more detail, which is configured to obtain spectral characteristics of an optical signal passing through water 10. Here, the sensor unit 120 comprises a measurement cell 125 that is arranged to be immersed (e.g., fully immersed) in water 10, e.g., when the sensor unit 120 is fully or partially submerged below the surface of the water 10. The measurement cell 125 is configured such that, in use, silt or the like is not accumulated significantly within the measurement cell 125. Here, the cell 125 comprises an open slotted arrangement 122 intended to be orientated downwardly relative to the water 10 (i.e., directed towards or otherwise facing towards the ground). In such a way, accumulation in the measurement cell 125 of silt or other debris entrained in the water is avoided, which may otherwise impede measurements. It will be appreciated that the sensor unit 120 in some examples may be adjustable or reconfigurable such that any signal path length across the cell 125 is adjustable (e.g., based on differing conditions).

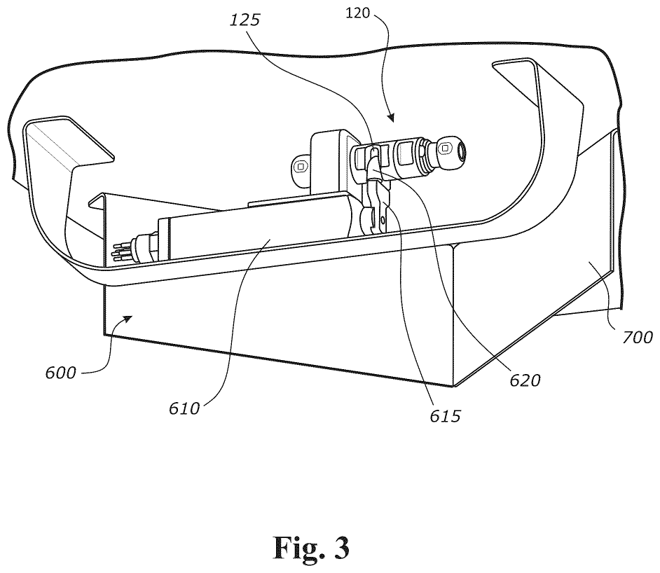

In some examples, the system 100 further comprises a cleaning arrangement 600 specifically configured to remove any debris or objects which may impede the light signal at the sensor unit 120 (e.g., within the measurement cell 125). FIG. 3 shows an isometric view of a portion of the system 100 comprising the sensor unit 120 arranged together with the cleaning arrangement 600. Here, the cleaning arrangement 600 is configured to pass through the measurement cell 125 so as to remove debris of the like (e.g., so as to remove debris within the cell or adhered to the walls of the cell). In this example, a swinging wiper arm 615 is configured to move a brush arrangement 620 through the slotted measurement cell 125. In use, a motor system 610 or similar may be used. The cleaning action, which leaves or otherwise parks a brush arrangement or other such cleaning arrangement, outside of the slot when not in use, may be performed from time to time, such as periodically, or as required (e.g., in the event of loss or reduced signal transmission). FIG. 3 further shows a guard arrangement 700, which is configured in the expected flow path of the sensor unit 120, upstream of the sensor unit 120.

Here, the sensor unit 120 is configured to permit measurement of water 10 within the cell using optical signals communicated across the measurement cell 125. Here, an optical measurement signal, as will be described below, is communicated from the link (e.g., fiber) connected to an input 127, across the measurement cell 125 (and through water 10), to an output 128, and returned to the link 115 for further transmission (e.g., communicated to an alternative return fiber for communicating the measurement signal onwards to the housing 110). As such, any measurement signal passes through the water 10 in the cell 125 and the spectra of that signal is influenced by the properties of the water 10, which is then usable to determine the parameters of those properties (e.g., nitrate composition).

Here, lens arrangements 129a, 129b are positioned either side of the measurement cell 125 so as to collimate a measurement signal across the cell 125 (e.g., and to and/or from the fiber of the link 115). In this example, each lens arrangement 129a, 129b has a particular axial focal length, i.e., a providing an axial position from the lens at which collimated light will be focused to a maximum intensity. Here, each lens 129a, 129b is positioned, in relation to respective fibers, such that the focal length is not collocated with the entry point of the light at the fiber. In that way, each lens 129a, 129b, may be considered defocused slighted from the ideal. While this may be considered to reduce the efficiency of the transmission of signal across the sensor unit 120, it nevertheless may permit a degree of tolerance in the event of mechanical variation of the sensor unit 120, e.g., optical alignment.

While in this example, the sensor unit 120 is configured to so that a measurement signal is transmitted across the cell 125 (i.e., once), from an input 127 to an output 128, it will be appreciated that in other examples, the input and output 127, 128 may be collocated, and that sensor unit 120 and cell 125 may be configured such that a measurement signal is transmitted and then reflected across the cell 125. In such a way, only a single communication path at the link 115 (e.g., single fiber) may be required to transmit and receive a measurement signal from the sensor unit 120.

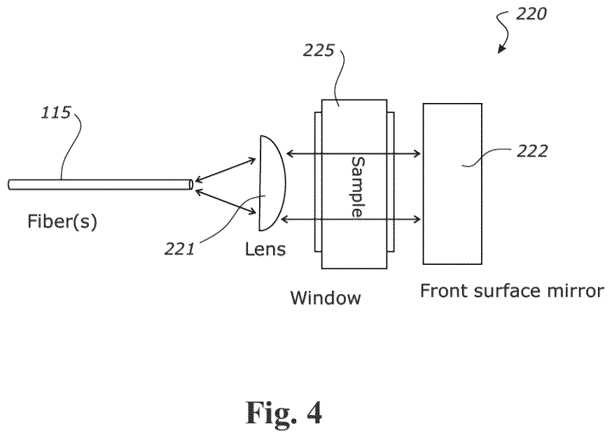

FIG. 4 shows an example of such an alternative sensor unit 220 with measurement cell 225. Here, a signal is communicated from the fiber/link 115 into a lens 221 which collimates the light through a cell 225 (and sample) whereupon it is reflected by a mirror 222, or the like, back through the sample, lens 221 and into the originating fiber/link 115 for return communication to the housing 110. Again, the lens 221 may be configured as described above.

In either event, the system 100 is configured such that the sensor unit 120, 220 is removably connectable to the housing 110. In that way, the communication link and/or the sensor unit 120, 220 may be replaced, for example when in damaged or on the basis of an alternative measurement set up, without having to modify or replace further components of the system 100. The housing 110 containing other components of the system 100 may be less susceptible to damage, and/or may comprise components usable across a range of alternative sensor units 120, 220 and/or lengths of communication link 115.

In some examples, the system 100 may be configured for use with multiple alternative sensor units and/or links, possibly of differing path lengths, etc. Such alternatives may be selectable by a user based on application. In such a way, in some cases the system 100 may be considered modular.

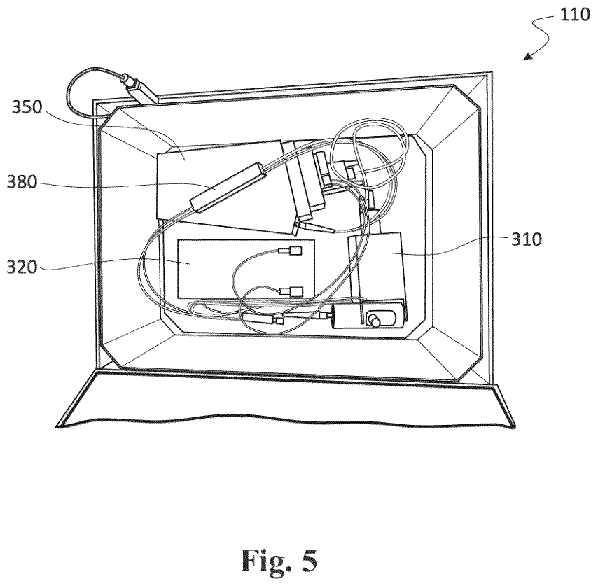

FIG. 5 show an example of the housing 110 of the system 100 in more detail. Here, the housing 110 provides environmental protection for further components of the system 100, and may be located remote from the water 10 (e.g., within 5 -15 meters, or so). The housing 110 may be accessible for service or maintenance purposes or the like. In this example, the housing 110 contains a light source 310, which is configured to provide a source signal, as well as a spectrometer 350, which are both operatively in communication with the sensor unit 120, 220, as will be described. As shown in FIG. 5, the system 100 further comprises an optical device 380 that is in communication with the light source 310 and is configured to provide a measurement signal and a reference signal from an emitted source signal. Here, the optical device 380 comprise an optical splitter as will be described in more detail below. Here, each optical component within the housing 110 is connected via optical cabling or the like. Each component may be selectively removed and replaced without the need to remove and/or replace further components. In that way, the system 100 may be considered to be modular and readily configurable for alternative set-ups as well as providing ease of maintenance.

An energy storage device 320 (e.g., battery) is also provided. Here, the storage device 320 is in communication with the power source 130 and can be configured to receive power (e.g., intermittent power) from that source 130 and store that energy at the housing 110 for use by the system 100. It will be appreciated that the power source 130 may be located further away from the housing 110 than, for example, the sensor unit 120, 220.

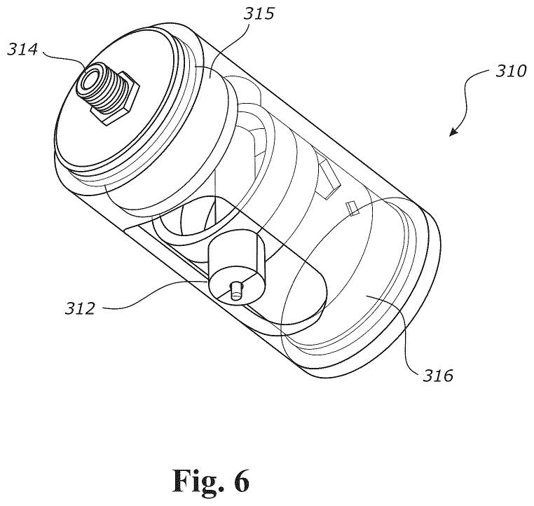

FIG. 6 shows an example of the light source 310 configured to provide a source signal. Here, the light source 310 is configured to provide a broadband signal, which is this example has a bandwidth comprising spectra between approximately 190 nm and 420 nm. It will be appreciated that this light source 310 itself may emit wavelengths beyond this bandwidth, but that the remainder of the system 100 (as will be described) is specifically configured to use and/or measure spectra within this range. In this particular example, the light source 310 is configured to provide a discrete period of optical output (e.g., flash) as opposed to a continuous output of source signal (in other examples a constant output may be provided).

The system 100 is configured such that measurements for use in determining properties of water are taken during those discrete periods of source signal emission (and may be taken over multiple emissions, e.g., and then averaged). Here, the light source 310 comprises flash tube 312 mounted within a housing of the light source 310. In this particular example, a xenon flash tube is used. The optical source 310 further comprises a mirror 316, which is a cylindrical mirror in this case, as well as a lens 315 arrangement configured to focus light from the flash tube 312 into a waveguide, e.g., optical fiber, at an output 314 of the source 310. In some examples, the spacing between two or more of the lens 315, flash 312 and mirror 316 may be adjustable (e.g., to help optimize the source signal).

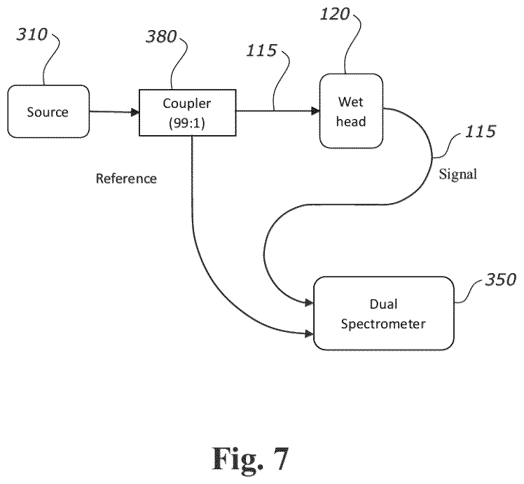

FIG. 7 shows a simplified schematic of the system 100 configured with a sensor unit 120 as per FIGS. 2a-2c, and including components within the housing 110. Here, the light source 310 is configured to emit the source signal, which is then split into a corresponding measurement signal and reference signals at the optical device 380. The measurement signal is communicated to the sensor unit 120, via the link 115, and transmitted across the measurement cell 125 (and water 10). The measurement signal is then communicated from the sensor unit 120 back to the housing 110 (and in particular to spectrometer 350) through a second fiber via link 115. The optical device 380 is configured such that the measurement signal and reference signal are similar in nature in so far as they contain spectral characteristics that are common, and common to the source signal (e.g., bandwidth and/or spectra). In this case, however, the optical device 380 is configured such that the overall power (e.g., intensity) of the measurement signal is much greater than that of the reference signal. Therefore, while the spectra of the measurement signal and reference signal may be the same or similar as they are communicated from the optical device 380, the intensity of the measurement signal may be greater. In this way, attenuation of the measurement signal that may occur along the link 115 can be accounted for such that, in use, the intensity of the measurement signal and reference signal is similar when received at the spectrometer 350.

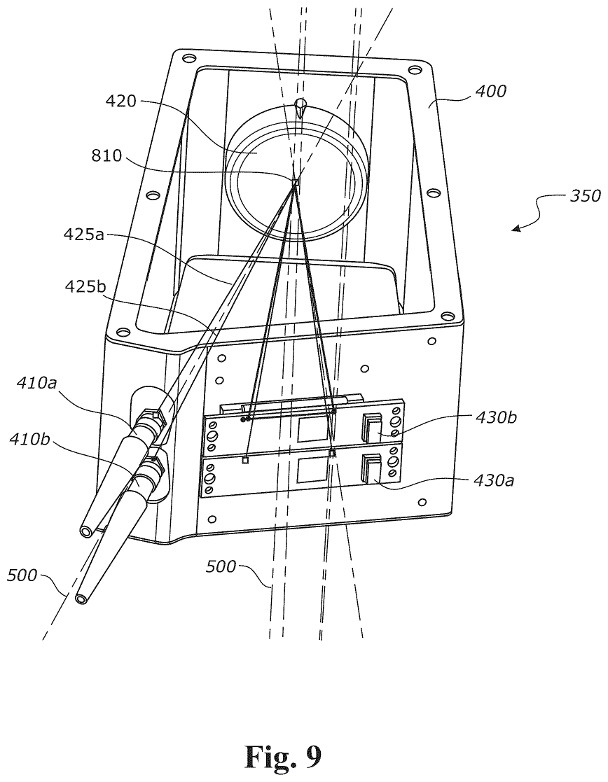

As such, in this example, the optical device 380 is configured to split the source signal such that more power is communicated towards the sensor unit 120, which is able to account for losses in the optical fiber as well as ensure that the signal is transmitted across the measurement cell (i.e., and not fully absorbed). The spectrometer 350 is configured to receive both the measurement signal (having been communicated across the measurement cell 125) as well as the reference signal that are associated with a particular emission (e.g., flash) from the light source 310. Those signals may be considered to be received at the spectrometer 350 at first and second channels 410a, 410b (see FIG. 9), and may be considered to be received simultaneously, or substantially simultaneously.

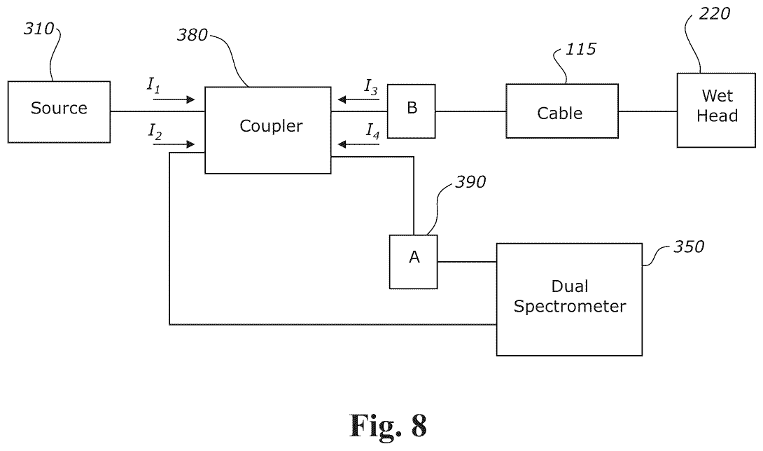

FIG. 8 shows an alternative simplified schematic of the system 100, which may be used together with sensor unit 220 shown in FIG. 4. As before, the optical device 380 is configured to split the source signal into measurement signal and reference signal with the measurement signal being communicated towards the sensor unit 220, and the reference signal being communicated to the spectrometer 350. In this case, however, the system 100 is configured such that the measurement signal is communicated to and from the sensor unit 220 using the same fiber in link 115 (e.g., transmitted to and/or from the sensor unit along a common fiber as both a forward and return path for the signal).

Here, the optical device 380 comprises an optical coupler (e.g., a 2×2 coupler), which in this example is provided with four ports, for example a first port 11, a second port 12, a third port 13, and a fourth port 14.

In this embodiment, the first port 11 is connected to light source 310 such that the optical device 380 is configured to split the light received from light source 310 into measurement signals and reference signals, whereby the measurement signal is communicated to the sensor unit 120 from the third port 13 and the reference signal (albeit now potentially also containing some of the measured signal) is communicated to the spectrometer 350 from the fourth port 14. Here, these two signals are approximately equal (e.g., equal power and/or intensity). Of course, it will be appreciated that the two signal are not required to be equal, as per the example given in FIG. 7. That said, proving a coupling ratio of at or around 1:1 (or 50:50) can help, and may optimize the power of the measurement signal for this arrangement. Here, the output from third port 13 is approximately equal to the output from fourth port 14. Additionally, in some cases, a relatively small fraction of the total source power will also propagate through port 12 by the nature of the optical coupler.

After the measurement signal has passed across the measurement cell 225 (e.g., through the analyte), the signal, which now exhibits some spectral changes due to absorption at the measurement cell 125, is communicated back through the link 115 toward to the optical device 380. The measurement signal is then communicated from the third port 13 of the device 380 to the second port 12 and with a significant decrease in intensity to port 14. Here, the measurement signal is also transmitted through the first port 106 back to optical source 102, however the impact of this may be considered negligible or otherwise by accommodated by the system 100.

As shown, the “measurement” signal, which contains spectral information from having been transmitted through the water 10, is then communicated from the second port 12 of the optical device 380 to the spectrometer 350 (e.g., a first channel 410a of the spectrometer 350). As mentioned, the “reference” signal is communicated from fourth port 14 of the optical device 380 to the spectrometer 350 (e.g., a second channel 410b of the spectrometer 350). In this case, the system 100 further comprise an attenuator 390 through which a reference signal passes, before reaching the spectrometer 350. In that way, the power of the reference signal can be attenuated so as to be commensurate with the power of a measurement signal being received at the spectrometer 350, e.g., and having been attenuated due to losses in the link 115 and/or when being communicated back through the optical device 380.

Here, signals at the second channel 410b of spectrometer 350 are able to be used to provide a spectral representation of a received reference signal, B=αR+bW, where R is the spectral signal of the source signal. Signals at the first channel 410a of spectrometer 350 are usable to provide a spectra representation of the “measurement” signal, A=εR+bW, where W is the spectral response of the analyte/water given the source, W=ζXR, and while α, b, β, ε and ζ are all constants, for a given frequency, and X is the spectral response of the analyte/water 10.

Linear algebra permits the separation of R and W from spectrometer responses from A and B. The system 100 then permits the calculation of X=W/ζR which provides a fully ratiometric measurement of the analyte spectra (eliminating the spectral characteristics of the source, which can vary from flash to flash). Then, via an absorbance calculation relative to a known standard analyte (or approximations or estimations of such an analyte), typically deionised water or the like, it is possible to calculate the nitrate concentration (e.g., correlating the absorption of particular wavelengths with calibration curve data for particular concentrations). It will be appreciated that this is possible even when the source light spectra changes from one flash to another. The analyte spectra calculation is much simpler for the system shown in FIG. 7, as B=cR and A=dW therefore the ratio R=A/B=eW produces the scaled (by a constant factor e) analyte spectra independent of the source spectra R. This ratiometric measurement allows the calculation of the absorbance and nitrate concentration as described previously, once again without the concern of flash-to-flash variation.

It will be appreciated that the optical device 380, when configured as an optical coupler, has the potential to provide a compact method to split the source signal and, unlike a free-space equivalent of the coupler (using partially reflective mirrors for example) the coupler has the potential to provide a much more stable and robust relationship between the various ports (and hence “constants”α, ε, and ζ remain “constant”).

In some further examples, it will be appreciated that a single spectrometer 350, e.g., a single input channel spectrometer 350, may be used in place of dual channel spectrometer 350.

Such a single spectrometer 350, e.g., with a mirror, or similar device, may be configured to select one or other path from the output of the optical device 380. Using multiple pulses, the single spectrometer can use a single fiber to calculate absorbance. This assumes that the source spectra is constant between the source light pulses. Where averaging is used, it is assumed that the source spectra is quasi-constant.

It will be appreciated however, that the spectra of the source signal may vary from flash to flash. This variation may be caused by many factors, including temperature and other such environmental conditions or inherent properties of the light source 310. While in some cases the variation may not be considered significant it nevertheless may cause problems in accurate measurement of properties of water 10, particularly when used at remote and/or exposed sites, or the like, and the composition of which is unknown and potentially comprises may species (e.g., complex nitrate and dissolved organic carbon compositions). In such examples, any unknown variation in the signal source spectra may have an impact of the assessment of the properties of the water 10.

In some examples, when comparatively assessing the absorption of the spectra of a measurement signal, it may not be suitable or accurate to simply using an indirect approximation of the spectra of the source signal. In order to derive a suitable accurate measurement, the specific spectra characteristics of the optical signal (e.g., using a reference signal) for many or each flash may be required, as has been described herein. It will be appreciated that such measurement may also allow accurate real time measurements to be taken, as well as accurate longer term trend to be established.

That said, however, there also remains a desire to have a spectrometer 350 arrangement that provides a simplified, compact and/or robust way to simultaneously measure both a measurement signal and a source signal for emissions or flash events so as to be able to accurately measure properties of the water.

Consider now FIG. 9, which shows one example the spectrometer 350 as used with the system 100 described herein. In this example, the spectrometer 350 may be considered to be a dual spectrometer. As described, the spectrometer 350 comprises a housing 400 and is configured to receive two optical inputs, here via first and second channel inputs 410a, 410b respectively (e.g., for measurement signal and reference signal). The spectrometer 350 further comprises a diffraction grating 420, which is configured to separate the two inputs into component wavelengths, or spectra, as will be described. Here, the spectrometer is configured such that diffraction of a bandwidth signal from first input provides the same wavelength components as the diffraction of the bandwidth signal from a second input. In that regard, the diffracted wavelength components may be common to similar bandwidth signals at both the first and second sensor arrays 430a, 430b. The specific common component wavelengths in this example are based on, or otherwise associated with, expected properties of the surface water and/or optical source signal, e.g., bandwidth of signal and expected absorption and/or transmission wavelengths.

The spectrometer 350 comprises a measurement sensor array 430a and a reference sensor array 430b, provided within the housing 400 and configured to receive common component wavelengths of measurement and reference signals having been diffracted from an illuminated grating 420. In this example, one or both of the sensor arrays 430a, 430b are adjustable at the housing 400, in that they can be moved at the housing 400 in order to permit refinement and calibration (e.g., for different operating conditions). The arrays may be adjustable relative to the wall of the housing (e.g., having two degrees of freedom, relative to the housing for example). In this example, the relative location of the diffraction grating 420 at the housing 400 is additionally or alternatively adjustable.

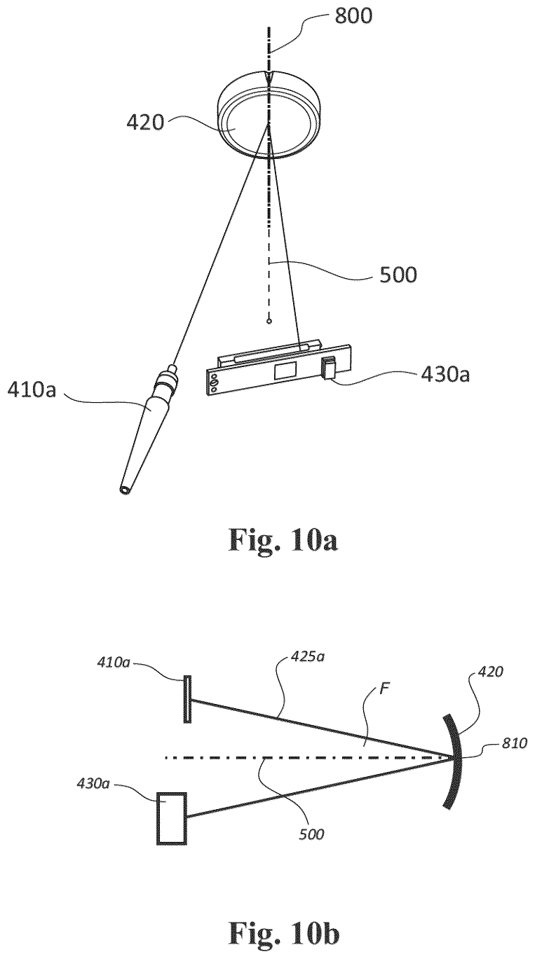

Here, the diffraction grating 420 is configured as a single grating, which in this example is a single concave grating 420. The grating 420 is further configured to be symmetrical. Here, the grating has an axis of symmetry 800 as shown in FIG. 10a. Here, the grating 420 has a principal optical focal plane 500. The principal optical plane 500 in this example is perpendicular to grating striations that are otherwise parallel with the axis of symmetry 800. The principal optical plane may be considered perpendicular to the nominal face of the grating 420.

The spectrometer 350 is configured such that the two optical inputs (e.g., derived from measurement signal and reference signal) are projected toward the grating 420 (e.g., from a slit, point source input, or in this example the fibers at the first and/or second channel inputs 410a, 410b, towards a middle region 810 or otherwise center point of the grating 420) along an off-axis focal line 510a, 510b. Here, the off-axis focal line 510a of the first input is different from an off-axis focal line 510b of second input. It will be appreciated that those signals may be projected as light cones towards the grating 420.

As such, this particular configuration produces a light cone projected towards the grating-the principal axis of which is pointed directly at the middle region 810 of the grating 420. Here, the spectrometer 350 is configured such that angle from the principal focal plane is approximately 11 degrees (e.g., an angle, α=11.4°). Here, the distance of 144.7 mm from the center is approximately 145 mm (e.g., 144.7 mm).

FIG. 10a shows an isometric view and 10b shows a corresponding side view of the diffraction grating 420 having the principal optical focal plane 500, and in which a first input is being projected towards the diffraction grating 420 along an off-axis focal line 425a. The off-axis focal line is defined by the angle F between the principal focal plane 500 and the line of projection 425a, shown in the side view in FIG. 10b. It will be appreciated that the second input is also projected as a cone towards the diffraction grating 420 at an angle as shown in FIG. 10a such that each of the wavelength components are diffracted back towards the respective measurement sensor array 430a at different positions along the sensor array depending on the particular frequency of the component wavelengths. In this example, the measurement sensor array 430a has a plurality of optical sensors configured to have a particular spectral resolution commensurate with the expected desired resolution of spectra. In this example, the arrays have a spectral resolution of approximately 1 nm (at least).

Returning now to FIG. 9, it can be seen that while the measurement signal is projected along a first off-axis focal line 425a, the reference signal is projected along a second off-axis focal line 425b, wherein the first and second off-axis focal line are positioned either side of the principal focal plane 500 of the grating 420 (e.g., equally at a common angle, either side of the principal plane). Similarly, the reference sensor array 430b is positioned in a corresponding manner at the other side of the principal optical plane 500 so as to receive wavelength components from the reference signal.

It will be appreciated that the spectrometer 350 (e.g., grating 420, etc.) essentially performs two simultaneous operations. The first operation is to split the incoming light into its component wavelengths. In other words, in this configuration, the spectrometer 350 (or grating 420, etc.) is configured to separate the incoming light (e.g., received measurement and/or reference signal) into a plurality or multiple component wavelengths (e.g., the ‘common component wavelengths’ referred to above) simultaneously. The second operation is to focus those components on a linear region within the housing (e.g., at the location of a corresponding array). In this particular example, incoming 200nm light is reflected to a point at an angle 5.5° from the normal and at a distance 133.1 mm from the grating center 810. The remainder of the light is focused along the line with increasing wavelength as you move back towards the input (e.g., entrance slit). The result is a focused set of rays which are split in wavelength from 200nm to 415nm (or the like) along a length of 25.4 mm. The sensor arrays described here have 2048 pixel each 0.2 mm high and 14 μm wide along this spectra (and it can be selected for their response to deep UV wavelengths).

It will be appreciated that using a single grating 520 in a spectrometer (e.g., dual spectrometer) is that where the inputs are stacked relative to one another above and below the principal optical plane 500 the grating 420, this may still generate a focused spectral line at an equal angle below the optical plane. This allows us to illuminate the grating 420 from two locations (above and below the optical plane) and then, without any interference between the two inputs, focus the respective spectra onto sensor arrays at an equal angle off-plane.

While this arrangement is shown for only two signals, it will be appreciated that in other examples, multiple further signals may be diffracted at further different angles from the principal plane 500. Further, while in this example the two inputs are projected along an off-axis focal lines that are positioned either side of the principal focal plane 500 of the grating 420, that need not always be the case, and in some examples, the offset focal lines may be offset on the same side of the principal focal plane 500, i.e., but at different offset angles. Such an arrangement would also have a differing configuration of sensor arrays 430a, 430b. A reader of ordinary skill in the art will readily be able to implement those alternatives accordingly.

In use, the spectrometer 350 described is able to produce two (or more) non-interfering spectra from two (or more) different inputs, e.g., a measurement signal and a reference signal, using use a single diffraction grating 420 (in this example a concave grating).

Properties of water, e.g., surface water, at site can be determined. It will further be appreciated that this may permit simultaneous measurement of a direct representation of the source signal (e.g., via the reference) and that of a measurement signal, which may allow the system 100 to account for differences in light output produced from one flash to another flash, when measuring water 10. In some cases, simultaneous in this regard may be considered to mean measurement over the duration of the flash, for example.

In any event, this improved approach may be of significant importance when considering the accuracy of the system 100, particularly when using components and an arrangement that are suitable for and used in varying environmental conditions, as well as when trying to measure surface water with an unknown and complex composition. It will be appreciated that this approach differs from, and provides more accuracy than, simply measuring an average output from the flash and potentially using that as a differential offset. Further, this system and method may be usable to determine a single target species or a combination of species in water, such as surface water (e.g., nitrate and/or dissolved organic carbon compositions). In some cases, the system 100 and/or method described may be considered to permit ratiometric measurement using both signals.

In some examples, where the output from the light source 310 (e.g., the spectra of the xenon flash) provides significantly more energy output at some wavelengths compared with others (or where a desired wavelength is of comparatively lower energy output), it may be helpful to filter the signal, or aspects of the signal (e.g., source signal, measurement signal and/or reference signal). This may mitigate or avoid issues such as clipping.

In some examples, the intensity of some component wavelengths may be reduced by using a filter (e.g., a short pass filter) at the first and second channel inputs 410a, 410b, for example. In this example, however, a non-wavelength selective filter (e.g., reflective neutral density (ND) filter) may be positioned over a portion of each of the sensor arrays 430a, 430b. In doing so, and by positioning such a filter in the appropriate location in front of the sensor arrays 430a, 430b, important component wavelengths may be communicated to the sensor arrays (such as at deep-UV for nitrates absorption) without loss, and other wavelengths may be attenuated and with careful choice of optical density signal clipping may be avoided.

In any event, it will be appreciated that the system 100 can be configured to measure the ratiometric difference in intensity between respective component wavelengths of a measurement signal and a reference signal (e.g., from the sensor arrays 430a, 430b) in order to determine properties in measured water 10. In some examples, calibration curve data may be used to help determine the properties of water.

In some examples, the system 100 may be configured to communicate data between the spectrometer and the remote site 160 (e.g., using the communication arrangement 150). In such cases, data processing and analysis may be performed remotely. In other examples however, the data processing may be formed locally at the housing 110 and/or system 100, and the water properties be communicated to remotely.

It has been identified that water at sites such as those described-particularly water found at the surface, such as in rivers or streams-may be affected significantly by varying environmental and geographic conditions. For example, varying weather conditions may have a significant bearing on the composition and turbidity of surface water at site. Molar absorptivity may be affected by such variations in the environment (e.g., temperature) as well as the time of measurement (e.g., season), even where the species is the same or generally the same. Further, the specific location may affect the composition of dissolved organic carbons, or the like, and this too may vary from location to location.

As such, in some cases, further data may be collected by the system 100 and/or communicated between a remote site and the system 100, such as location-based data, environmental data (e.g., temperature, etc.), temporal data (e.g., time of day and/or month and/or year, or other seasonal data), water matrix data, etc. Such additional data may be stored and used to provide calibration data for the system 100 (e.g., calibration curve data). In such cases, the system 100 may be configured to use calibration data comprising one or more of: environmental data, temporal data, location-based data, in order to determine properties of measured water.

The system 100 may comprise a calibration database, which may contain multiple calibration dataset for differing conditions. The database may be stored at the housing, for example, or remotely, and may be usable together with the measurements from the system 100 in order to determine properties of measured water. A reader of ordinary skill in the art will readily be able to implement those various alternatives.

Changes and modifications in the specifically described examples can be carried out without departing from the principles of the present disclosure which is intended to be limited only by the scope of the appended claims, as interpreted according to the principles of patent law including the doctrine of equivalents.

1. A system for measuring properties of water at site, the system comprising:
a light source configured to emit a broadband source signal comprising a particular bandwidth for use in measuring a range of particular properties of water;
an optical device configured to split the source signal into a measurement signal and a corresponding reference signal;
a sensor unit configured to communicate the measurement signal through water at the sensor unit; and
a spectrometer configured to receive both the measurement signal having been communicated through water at the sensor unit and the reference signal from the optical device, the spectrometer configured to separate the received measurement signal and the reference signal into common component wavelengths, the common component wavelengths being associated with expected properties of water;
wherein the system is further configured to use respective ones of the component wavelengths of the measurement signal together with those of the reference signal to determine one or more particular properties of measured water.

2. The system according to claim 1, wherein the spectrometer comprises a diffraction grating and wherein the diffraction grating is configured to separate both the received measurement signal and the received reference signal into their component wavelengths.

3. The system according to claim 2, wherein the diffraction grating is configured as a concave grating being symmetrical and having a principal optical focal plane, and wherein the received measurement and reference signals are projected toward the diffraction grating along respective off-axis focal lines, the off-axis focal line of the measurement signal being different from the off-axis focal line of the reference signal.

4. The system according to claim 3, wherein the spectrometer is configured such that the measurement signal is projected along a first off-axis focal line, and the reference signal is projected along a second off-axis focal line, and wherein the first and second off-axis focal lines are positioned on either side of the principal focal plane of the diffraction grating.

5. The system according to claim 3 wherein the spectrometer comprises a measurement sensor array and a reference sensor array, wherein the measurement and reference sensor arrays are configured to receive the common component wavelengths of the measurement and reference signals having been diffracted from the diffraction grating.

6. The system according to claim 5, wherein the sensor arrays are positioned off-axis from the principal plane of the diffraction grating.

7. The system according to claim 6, wherein one or both of the sensor arrays are adjustable relative to the principal plane of the diffraction grating.

8. The system according to claim 7, wherein the diffraction grating is adjustable so as to provide the adjustment relative to the principal plane.

9. The system according to claim 5 wherein the spectrometer comprises a filter arrangement positioned over portions of the measurement sensor array and the reference sensor array, the filter arrangement configured to filter some of the component wavelengths of the measurement and reference signals received at the sensor arrays.

10. The system according to claim 1 configured such that the spectrometer receives both the measurement signal and the reference signal simultaneously for a particular emission from the light source.

11. The system according to claim 1 configured such that the spectrometer separates at least one of the received measurement signal and the received reference signal into a plurality of the component wavelengths simultaneously.

12. (canceled)

13. The system according to claim 1 configured such that the spectrometer separates the received measurement signal and reference signal into a plurality of the component wavelengths simultaneously.

14. The system according to claim 1 wherein the sensor unit comprises a slotted gap, within which water is located in use, and through which the measurement signal is communicated, and wherein the sensor unit is configured such that an opening of the slotted gap is directed downwardly when positioned in water, in use.

15. (canceled)

16. (canceled)

17. The system according to claim 1, wherein the optical device is configured as an optical coupler.

18. The system according to claim 1, configured to use measured component wavelengths of the measurement signal and the reference signal, together with ancillary data, in order to determine properties of measured water.

19. The system according to claim 18, wherein the ancillary data comprises data associated with at least one of location of the system, a time of measurement, a temperature, and properties of a measured water matrix.

20. The system according to claim 18, wherein the system comprises, or is in communication with, a database comprising a plurality of calibration datasets for use in determining properties of measured water based on the measurement and reference signals, and wherein the system is configured to select one or more particular calibration datasets for use, based on the ancillary data.

21. (canceled)

22. (canceled)

23. The system according to claim 1, wherein the particular bandwidth comprises a wavelength band between 190 nm and 420 nm.

24. A method for measuring properties of water at site, the method comprising:
emitting a broadband source signal comprising a particular bandwidth for use in measuring a range of particular properties of water;
splitting the source signal into a measurement signal, and a corresponding reference signal;
communicating the measurement signal through water;
receiving both the measurement signal, having been communicated through water, and the reference signal, and separating the measurement signal and reference signal into common component wavelengths, the common component wavelengths being associated with expected properties of water; and
using respective ones of the component wavelengths of the measurement signal together with those of the reference signal to determine one or more particular properties of measured water.

25. (canceled)

26. (canceled)

27. (canceled)

28. (canceled)

29. (canceled)

30. (canceled)

31. (canceled)

32. (canceled)

33. (canceled)

34. A system for measuring properties of water at site, the system comprising:
a light source configured to emit a broadband source signal comprising a particular bandwidth for use in measuring a range of particular properties of water;
a sensor unit configured to receive via a communication fiber, from the light source, a measurement signal and communicate the measurement signal through water at the sensor unit, wherein the sensor unit is configured to reflect the measurement signal back across water for receipt and further transmission using the communication fiber; and
a spectrometer configured to receive a measurement signal from the sensor unit, and to separate that measurement signal into component wavelengths, the component wavelengths being associated with expected properties of water;
wherein the system is further configured to use the component wavelengths of the measurement signal to determine one or more particular properties of measured water.

35. (canceled)

36. (canceled)

37. (canceled)

38. (canceled)
